(19) United States
(12) Patent Application Publication
(10) Pub. No.: US 2021/0125729 A1
(43) Pub. Date: Apr. 29, 2021

(54) DONOR ENGAGEMENT SOLUTION

(71) Applicant: Delcon Inc., Charlotte, NC (US)

(72) Inventor: Gerald Ragozine, Mt. Pleasant, SC (US)

(73) Assignee: Delcon USA, Inc., Charlotte, NC (US)

(21) Appl. No.: 17/078,454

(22) Filed: Oct. 23, 2020

Related U.S. Application Data
(60) Provisional application No. 62/925,526, filed on Oct. 24, 2019.

(51) Int. Cl. G16H 50/70; H04L 29/08; G16H 10/60; G06K 19/07; G06K 7/10; G06Q 50/00; G06Q 40/02; G06F 16/22; G06F 9/451

(52) U.S. Cl. CPC G16H 50/70; H04L 67/36; H04L 67/12; G16H 10/60; G06K 19/0723; G06K 2007/10504; G06Q 50/01; G06Q 40/02; G06F 16/22; G06F 9/451; G06K 7/10297

(57) ABSTRACT

A method for engaging blood donors through an Internet computer network by customizing contents on a user interface of a mobile communication device or a computer according to each blood donor includes:
providing a database on a remote server configured to store a plurality of information of a plurality of blood donors;
providing a physical token for each blood donor, each physical token storing a unique identifier of the blood donor;
reading the unique identifier from the physical token, and transmitting the unique identifier to the database through the Internet;
comparing, by a processor, the transmitted unique identifier with the plurality of unique identifiers stored on the database to verify the blood donor's identity;
retrieving the plurality of information stored on the database associated to the transmitted unique identifier;
updating the user interface with the plurality of information stored in the database and associated to the transmitted unique identifier.

### CROSS REFERENCE TO RELATED APPLICATIONS

This application claims the benefit of U.S. Provisional Application No. 62/925,526, filed Oct. 24, 2020, which is incorporated by reference as if fully set forth.

### FIELD OF INVENTION

The present invention concerns a blood donor engagement solution that allows blood centers to engage donor and potential donors more easily. One aim of the present invention is also to increase the commitment of any citizen with respect to the increasing relevance and need of donating blood.

### BACKGROUND

Healthcare providers continually face a need for donated blood to treat patients. Blood donation organizations often have difficulty in notifying and educating the public regarding donations. As a result, donors may not become repeat donors due to a lack of communication, information and access since current protocols are cumbersome and outdated.

### SUMMARY

According to a first aspect of the present invention, a method is provided for engaging blood donors through an Internet computer network by customizing contents on a user interface of a mobile communication device or a computer according to each blood donor. The method comprises:
providing a database on a remote server configured to store a plurality of information of a plurality of blood donors, the information concerning each blood donor being associated to a unique identifier of the blood donor,
providing a physical token for each blood donor, each physical token storing a unique identifier of the blood donor,
reading with a user device the unique identifier from the physical token, and transmitting the unique identifier to the database through the Internet computer network,
comparing, by a processor, the transmitted unique identifier with the plurality of unique identifiers stored on the database to verify blood donor identity,
retrieving the plurality of information stored on the database associated to the transmitted unique identifier,
updating the user interface presented at the user device with the plurality of information stored in the database and associated to the transmitted unique identifier.

The plurality of information of a blood donor may include an identification code of the user device of the blood donor.

In an alternate embodiment, the method may also include:
transmitting the identification code of the user device together with the unique identifier read from the physical token to the database through the Internet computer network,
comparing, by a processor, the transmitted unique identifier and the identification code of the user device with the unique identifier and the identification code of the user device stored in the database,
updating the user interface presented at the user with a request to subscribe to a blood donation campaign if the transmitted identification code is different from the identification code stored in the database.

According to a second aspect of the present invention, a system is provided for engaging blood donors. The system comprises:
a server connected to a network, the server comprising:
at least one processor, a database for storing information, and a memory operatively coupled to the processor,
the database being configured to store a plurality of information of a plurality of blood donors, the information concerning each blood donor being associated to a unique identifier of the blood donor,
the memory further storing program instructions that when executed by the processor, causes the processor to:
analyzing the information concerning the plurality of blood donors,
identifying a group of blood donors having the same type of predetermined information among the plurality of information of each blood donor,
transmitting predetermined contents to a user device of each of the blood donor of the group identified.

According to a third aspect of the present invention, a physical token is provided for engaging blood donors through an Internet computer network, the physical token comprising a wearable device containing a machine-readable medium wherein a unique identifier of a blood donor is stored. The physical token may also include an RFID tag as the machine-readable medium.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 is a schematic diagram of a system for engaging blood donors, in accordance with the present invention;

FIG. 2 is schematic diagram of a detail of the system of FIG. 1;

FIG. 3 is a graphical representation of features of the system of FIG. 1;

FIG. 4 is a graphical representation diagram of a method in accordance with the present invention;

FIG. 5 is a graphical representation of a user interface being customized;

FIG. 6 is a graphical representation of a user post on social media; and

FIG. 7 is a flow diagram of the method of the present invention.

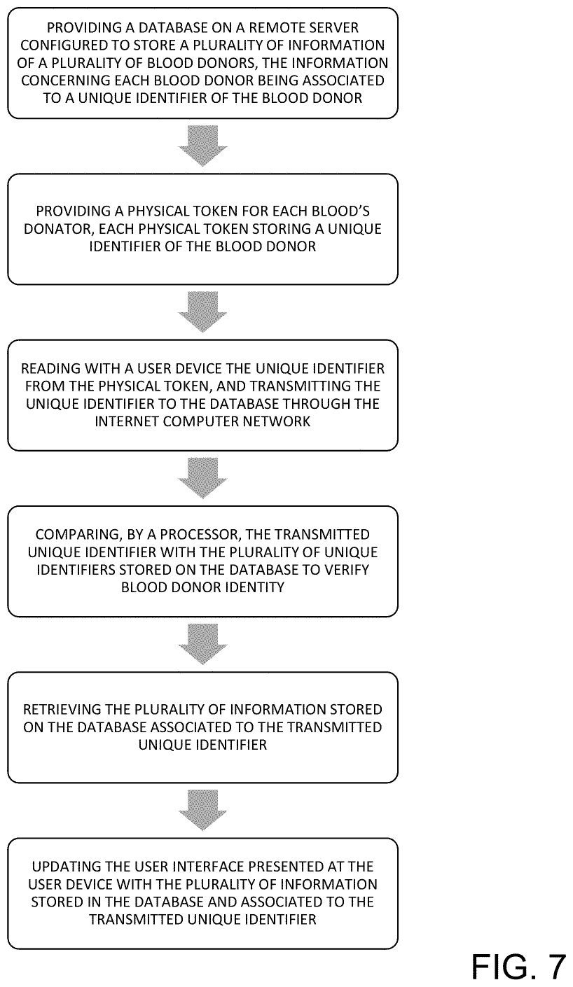

### DETAILED DESCRIPTION OF THE PREFERRED EMBODIMENTS

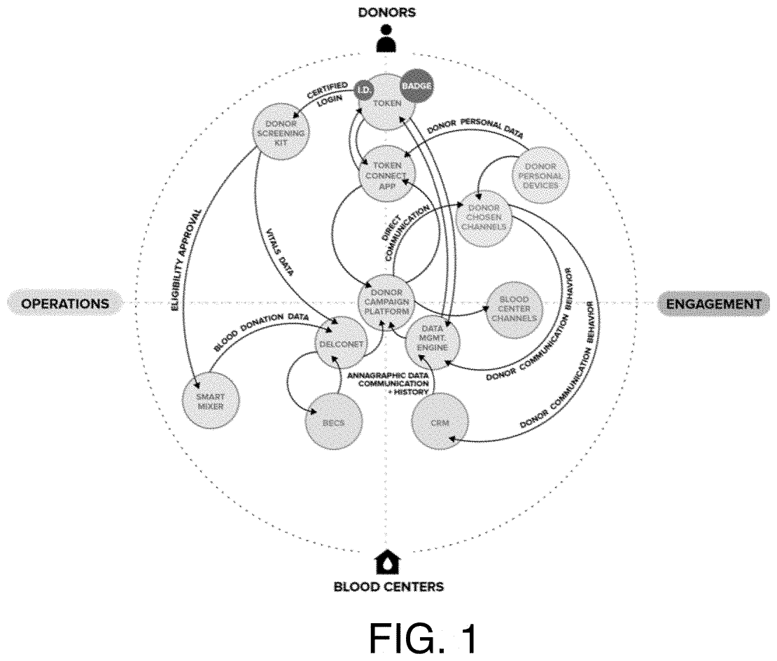

The framework of any blood donating ecosystem substantially comprises from one side the blood centers and from the other side the donors. As schematically shown in FIG. 1, the blood centers generally have their primary engagement channels such as website, app, instant messaging, and social media touchpoints where each blood center is present. At the same time, the blood centers also adopt software that help companies to manage interactions with new and existing customers, and helps organizations streamline their processes, build relationships, etc., such as for example, Customer Relationship Management (CRM) software. Moreover, blood centers adopt software to detect disease or other conditions in donors, or preventing disease by stopping the release of unsuitable blood and blood components, such as for example, Blood Establishment Computer Software (BECS).

At the same time, the donors already have donor chosen channels, i.e. the communication channels the donor chooses to use for direct communication with the blood centers (e.g. Facebook Page, Instagram, WhatsApp, SMS, email, etc.). The donors usually use donor personal devices, i.e. the physical devices donors use to access their chosen communication channels, for example, smartphone, tablet, personal computers, etc.

An aim of the present invention is to enhance the relationships between the blood centers and donors in a blood donating ecosystem.

In order to reach this goal, the present invention comprises a turnkey marketing solution that allows blood centers to easily engage donors and potential donors in a meaningful conversation by customizing contents through an online Donor Campaign Platform™, while leveraging social media that the donor already participates in.

With reference still to FIG. 1, the present method and system include hardware and physical devices:

Physical Donor Token

It is a physical token connecting the digital and physical world. A unique giveaway device for improving identification and customer care at the donation site. It allows the donor to log into the platform, in the system and/or on specific websites. The Physical Donor Token includes RFID and/or QR code for easy connection to the digital world, for example through a phone camera or NFC (near field communication) reader of the user.

The Physical Donor Token can also facilitate identification and additional safety monitoring with potential future add on options. By wearing the Donor Token™ received at blood donation out into the community the Ambassador is displaying a physical sign of the value and honor of being a blood donor. This act supports an organic marketing campaign as the token is seen by or shown to others. A natural conversation starter, interested parties can then easily access the Ambassador Program™ by scanning the token with their own phone, thus activating a communication line with the blood center welcoming a new member to the community.

Both Donor Tokens provide improved connection between blood centers and donors without the need for a proprietary app and costs and challenges associated with maintaining an app. Instead leveraging on social media and apps the donor is already using.

Both Donor Tokens serve as a «badge of honor», a social statement worn by the donor, fostering conversations in the community.

Donor Screening Kit

An automated device for semi-autonomous blood donation eligibility check.

Smart Mixer/Scales

A blood pouch scale that has smart features to facilitate operators, improve donation process and seamlessly gather data about it.

Data Management Engine

The back-end system needed to support the management of data streams to and from the digital platforms. It enables Blood Centers to deliver more targeted communication campaign and engagement strategies.

With reference still to FIG. 1, the present invention comprises the following software.

Social Donor Token

It is a unique digital giveaway, i.e. on a digital platform, that provides blood centers with an extra touchpoint for donor engagement, opening up opportunities for new engagement strategies and data gathering.

Token Connected App

A ‘control panel’ app to activate the synchronization with third-party apps and devices, and manage the functionalities of the token.

Donor Campaign Platform

A software for promotion, engagement and retention provides a seamless way to customize and send out marketing campaigns by meeting the specific needs of blood centers.

Middleware Software

A middleware that integrates data received from smart scales with the BECS system of the blood center. In the future it might be able to collect data from all of blood collection devices (screening kit, token ID, etc.).

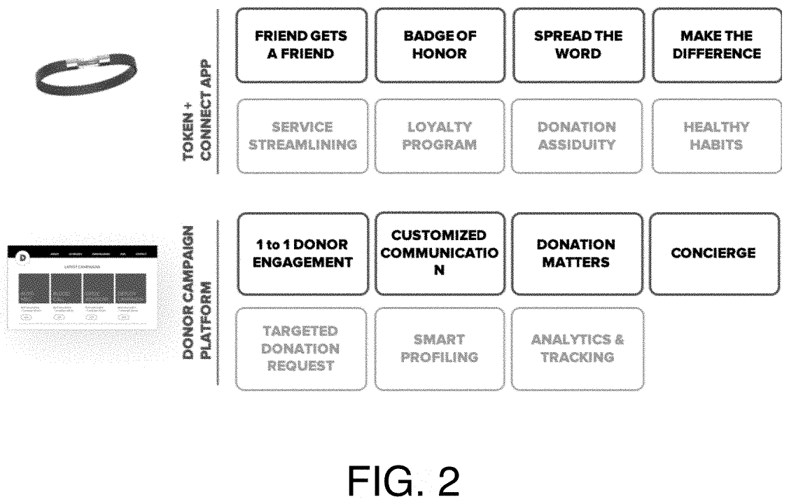

The blood donor engagement solution also transmits and exchanges a list of priority features between the blood centers and donors. For example, with reference to FIG. 2:

Friend Gets a Friend

A method enabling donors to recruit new donors in the physical world so to help blood centers in acquiring prospects. The goal of this method is to streamline service.

Badge of Honor

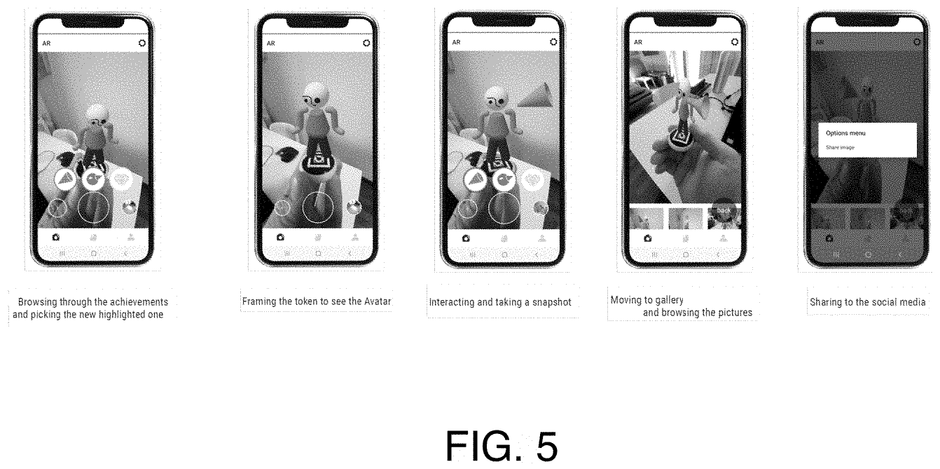

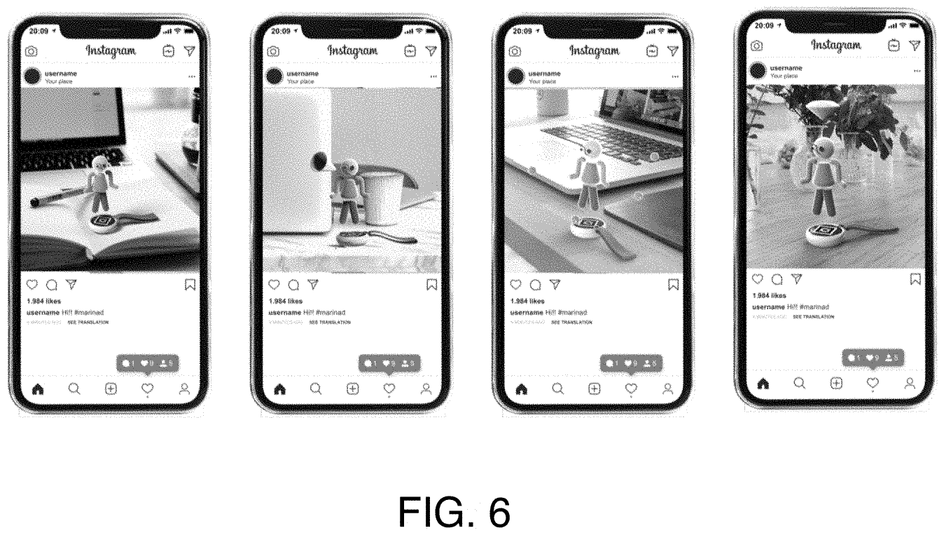

A method for motivating donors by leveraging social recognition and enabling them to show their commitment. The more a Donor Ambassador™ donates blood and/or recruits new ambassadors the higher status he achieves. This can be signified with different colors, or other creative ways to indicate an ambassador's status and possibly qualify for rewards. The goal of this method is to promote a loyalty program. FIG. 5 shows some exemplary screen shots of a smart device of a donor, disclosing the choice of different features of the avatar obtained through the achievement of an higher status in the donor campaign, the framing of the token to see the new upgraded avatar, the interaction with the new feature and the pick of a snapshot of the avatar having the new feature, the moving of the upgraded avatar to a gallery in order to browsing the pictures, and sharing on the picture on social media. In FIG. 6 are shown some exemplary screen shots of a smart device of a donor, disclosing the sharing on a photo and video sharing social networking service of different pictures obtained through the previously disclosed steps shown in FIG. 5.

Spread the Word

A method for turning donors into spokespersons of the blood center to increase commitment and recruitment. The goal of this method is to increase donation assiduity.

Make the Difference

A method for keeping donors' commitment high by sharing with them the achievements of blood center's campaigns. The goal of this method is to increase healthy habitudes.

1 to 1 Donor Engagement

A method for opening up and automatize communication on the donors' favorite/frequent digital channel. The goal of this method is to obtain targeted donation requests.

Customized Communication

A method for empowering donation ambassadors to create “rich media” content for p2p communication campaigns. The goal of this method is to reach a smart profiling.

Donation Matters

A method for educating about the importance of donating while delivering suggestions for being a healthy donor. The goal of this method is to set up analytics and tracking.

Concierge

A method comprising extra services for donors, such as reminders of appointments, or info about how to reach the blood center.

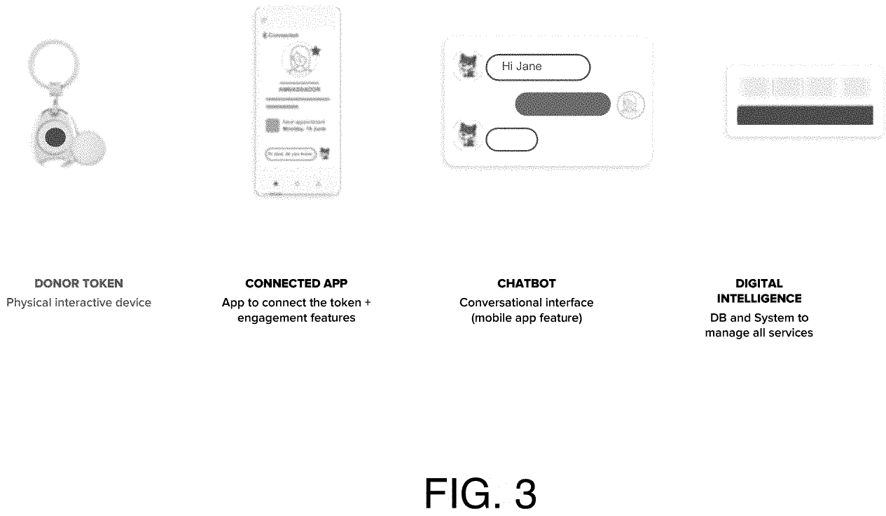

According to one the possible embodiments of the present invention shown in FIG. 3, the donor engagement solution comprises a DONOR TOKEN, i.e. a physical interactive device, a CONNECTED APP, i.e. an app to connect the token plus engagement features, a CHATBOT, i.e. conversational interface (mobile app feature), and a DIGITAL INTELLIGENCE, i.e. a data base and a system to manage all services.

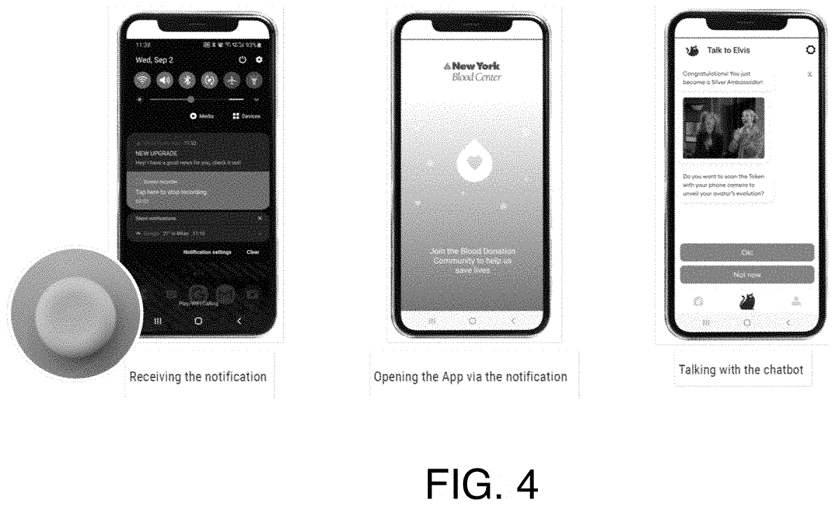

FIG. 4 shows some exemplary screen shots of a smart device of a donor, disclosing the receiving of a notification from a blood center, the opening of the app directly from the notification, and the start of a conversation with the chatbot of the blood center.

The Donor Token as a low-cost device has the main purpose to activate a direct communication channel with the donor. Further strategies to keep the donor engaged in using the device can be added by leveraging the Connect App.

Connecting the physical and digital world provides a solution which allows to target and engage younger generations, the involvement of this target it is currently a concern of blood center. Conveying the pride of being a donor both on social media and through tangible feedback.

The Chatbot and the Digital Intelligence shall ac as a concierge service for donors which guides them during the different phases of the donor experience and delivers relevant and engaging information meant to nurture the relationship with the donor.

Educating, sensitizing and motivating youngsters by upgrading the system's functionalities to make interactions more fun and leveraging gamification.

Informing about the importance of donating blood while leveraging social recognition (i.e.: one donation can save a life, this year you have saved 3 lives).

Several functionalities may also be added to the donor engagement solution. For example, TOKEN TRIGGER, the Donor Token serves to prompt the system to perform a certain action (i.e.: link share, virtual object placement, etc.). TOKEN FEEDBACK, the token serves as tangible output to notify users when the chatbot wants to talk to them (i.e.: info about traffic, new campaigns). TOKEN AWARENESS SURFACE, the token shall show a certain status symbolizing and reacting to the commitment to the donation cause (i.e.: ambassador/campaign status). APP INPUT.

the app allows to open up a direct communication channel and gather extra data about their profile. APP OUTPUT, the app provides rich media content, to increase engagement in the interaction and communication (visuals, conversation).

QUESTIONING, the chatbot involves the user in the conversation to find out more about them (profiling) and uses a quiz their level of awareness about donation. INFORMING, the chatbot fosters commitment through information about being a healthy donor, ongoing campaigns and Ambassadors profile status. ENGAGING, the chatbot proactively starts a conversation with donors to keep the customer relationship going and trigger their advocacy for the cause. CONTENT EDITING, the Digital Intelligence system allows to manage all conversational flows and editorial contents with a conversational editor and also includes the content repository for all kind of virtual objects and images. BACKEND SYSTEM, the system allows the management of the entire logic and integrates available data from middleware software, Blood Center system (CRM, . . . ) and donor profile data.

Other possible services shall be quiz chat, new donor campaign advertisements, token interaction and send a postcard to invite a friend using the smartphone's camera.

As well as Quick Onboarding, the donor receives the token at the blood donation site thus consenting to receive communications from the Blood Center. The Donor Ambassador Program™ is activated by scanning the token with the donor's phone thus opening up a line of communication through existing social media.

According to one of the embodiments of the present invention the Donor Token Physical might comprise a wearable device, for example a wristband, or a keychain, or a necklace, comprising an integrated RFID or QR code for interacting with other devices and enhancing marketing opportunities.

The QR Code shall be used as a simple trigger for action: activation of the Donor Ambassador Loyalty Program™, opt-in for message receiving, engaging friends . . .

The Donor Token Physical might be manufactured in different colors for different purposes. For example, to assign a “badge of honor” to recurring donors and donation champions: the color is used to indicate the achievement level of each donor i.e. number of donations, number of donors recruited.

The Donor Token Physical might also be used to identify different blood types or to identify “special donors” i.e. eligible for a specific blood product or requiring special attention.

The blood donor engagement solution shall be developed on a system comprising an Internet computer network, mobile communication devices or computers each comprising a user interface, a server connected to the computer network, a database on the server configured to store a plurality of information, a processor operatively coupled to a memory, and a physical token for a blood donor.

It is understood, therefore, that this invention is not limited to the particular embodiments disclosed, but is intended to cover all modifications which are within the spirit and scope of the invention as defined by the appended claims; the above description; and/or shown in the attached drawings.

1. A method for engaging blood donors through an Internet computer network by customizing contents on a user interface of a mobile communication device or a computer according to each blood donor, the method comprising:
providing a database on a remote server configured to store a plurality of information of a plurality of blood donors, the information concerning each blood donor being associated to a unique identifier of the blood donor,
providing a physical token for each blood donor, each physical token storing a unique identifier of the blood donor,
reading with a user device the unique identifier from the physical token, and transmitting the unique identifier to the database through the Internet computer network,
comparing, by a processor, the transmitted unique identifier with the plurality of unique identifiers stored on the database to verify blood donor identity,
retrieving the plurality of information stored on the database associated to the transmitted unique identifier,
updating the user interface presented at the user device with the plurality of information stored in the database and associated to the transmitted unique identifier.

2. The method of claim 1, wherein the plurality of information of a blood donor comprises an identification code of the user device of the blood donor.

3. The method of claim 2, wherein the method further comprises:
transmitting the identification code of the user device together with the unique identifier read from the physical token to the database through the Internet computer network,
comparing, by a processor, the transmitted unique identifier and the identification code of the user device with the unique identifier and the identification code of the user device stored in the database,
updating the user interface presented at the user with a request to subscribe to a blood donation campaign if the transmitted identification code is different from the identification code stored in the database.

4. A system for engaging blood donor comprising:
a server connected to a network, the server comprising:
at least one processor, a database for storing information, and a memory operatively coupled to the processor,
the database being configured to store a plurality of information of a plurality of blood donors, the information concerning each blood donor being associated to a unique identifier of the blood donor,
the memory further storing program instructions that when executed by the processor, causes the processor to:
analyze the information concerning the plurality of blood donors,
identify a group of blood donors having the same type of predetermined information among the plurality of information of each blood donor,
transmit predetermined contents to a user device of each of the blood donor of the group identified.

5. A physical token for engaging blood donors through an Internet computer network, the physical token comprising a wearable device containing a machine-readable medium wherein a unique identifier of a blood donor is stored.

6. The physical token of claim 5, wherein the machine-readable medium is an RFID tag.
